(19) United States
(12) Patent Application Publication
(10) Pub. No.: US 2018/0186734 A1
(43) Pub. Date: Jul. 5, 2018

(54) PROCESSES FOR THE PREPARATION OF PESTICIDAL COMPOUNDS

(71) Applicant: Dow AgroSciences LLC, Indianapolis, IN (US)

(72) Inventors: Patrick HEIDER, Midland, MI (US); Kaitlyn GRAY, Indianapolis, IN (US); Patrick T. MCGOUGH, Midland, MI (US); Jayacandran DEVARAJ, Midland, MI (US); Mark E. ONDARI, Midland, MI (US)

(21) Appl. No.: 15/853,083

(22) Filed: Dec. 22, 2017

Related U.S. Application Data
(60) Provisional application No. 62/440,234, filed on Dec. 29, 2016.

(51) Int. Cl. C07C 319/18; C07C 319/28; A01N 37/36; C07C 323/52

(52) U.S. Cl. CPC C07C 319/18; C07C 323/52; A01N 37/36; C07C 319/28

(57) ABSTRACT

The disclosure relates to efficient and economical synthetic chemical processes for the preparation of pesticidal thioethers. Further, the disclosure relates to certain novel compounds useful in the preparation of pesticidal thioethers.

### CROSS-REFERENCE TO RELATED APPLICATIONS

This application claims priority under 35 U.S.C. § 119(e) to U.S. Provisional Application Ser. No. 62/440,234 filed Dec. 29, 2016, which is incorporated herein by this reference in its entirety.

### TECHNICAL FIELD

This application relates to efficient and economical synthetic chemical processes for the preparation of pesticidal thioethers. Further, the present application relates to certain novel compounds useful in the preparation of pesticidal thioethers.

### BACKGROUND

There are more than ten thousand species of pests that cause losses in agriculture. The worldwide agricultural losses amount to billions of U.S. dollars each year. Stored food pests eat and adulterate stored food. The worldwide stored food losses amount to billions of U.S. dollars each year, but more importantly, deprive people of needed food. Certain pests have developed resistance to pesticides in current use. Hundreds of pest species are resistant to one or more pesticides. The development of resistance to some of the older pesticides, such as DDT, the carbamates, and the organophosphates, is well known. However, resistance has even developed to some of the newer pesticides. As a result, there is an acute need for new pesticides that has led to the development of new pesticides. Specifically, US 20130288893(A1) describes, inter alia, certain pesticidal thioethers and their use as pesticides. Such compounds are finding use in agriculture for the control of pests.

Because there is a need for very large quantities of pesticides, specifically pesticidal thioethers, it would be advantageous to produce pesticidal thioethers efficiently and in high yield from commercially available starting materials to provide the market with more economical sources of much-needed pesticides.

### BRIEF DESCRIPTIONS OF THE DRAWINGS

FIG. 1 is a plot of temperature vs. time for a reaction comprising benzoyl peroxide and either 4, N,N-trimethylaniline or N,N-dimethylaniline.

FIG. 2 is a diagrammatic view of a semi-batch process in accordance with the present disclosure, showing the different reagent feedstreams that may be added to the reaction vessel.

FIG. 3 is a diagrammatic view of a continuous process in accordance with the present disclosure, showing from left to right the reactants, an optional catalyst activation reactor, a loop reactor, and a finishing reactor.

FIG. 4 is a diagrammatic view of a purification procedure in accordance with the present disclosure, showing the crude material may enter a reboiler of a first distillation apparatus, and further showing a fraction provided by the first distillation apparatus can be further purified in a second distillation apparatus.

### DEFINITIONS

As used herein, the term “alkyl” includes a chain of carbon atoms, which is optionally branched including but not limited to C₁-C₆, C₁-C₄, and C₁-C₃. Illustrative alkyl groups include, but are not limited to, methyl, ethyl, n-propyl, isopropyl, n-butyl, isobutyl, sec-butyl, tert-butyl, pentyl, 2-pentyl, 3-pentyl, and the like. Alkyl may be substituted or unsubstituted. It will be understood that “alkyl” may be combined with other groups, such as those provided above, to form a functionalized alkyl. By way of example, the combination of an “alkyl” group, as described herein, with a “cycloalkyl” group may be referred to as an “alkyl-cycloalkyl” group.

As used herein, the term “cycloalkyl” refers to an all-carbon cyclic ring, optionally containing one or more double bonds but the cycloalkyl does not contain a completely conjugated pi-electron system. It will be understood that in certain embodiments, cycloalkyl may be advantageously of limited size, such as C₃-C₆. Cycloalkyl may be unsubstituted or substituted. Examples of cycloalkyl include cyclopropyl, cyclobutyl, and cyclohexyl.

As used herein, the term “aryl” refers to an all-carbon cyclic ring containing a completely conjugated pi-electron system. It will be understood that in certain embodiments, aryl may be advantageously of limited size, such as C₆-C₁₀. Aryl may be unsubstituted or substituted. Examples of aryl include phenyl and naphthyl.

As used herein, “halo”, “halogen”, or “halide” may be used interchangeably and refers to fluorine (F), chlorine (Cl), bromine (Br) or iodine (I).

As used herein, “hydroxy” or “hydroxyl” refers to an —OH group.

As used herein, “trihalomethyl” refers to a methyl group having three halo substituents, such as a trifluoromethyl group.

### DETAILED DESCRIPTION

The compounds and process of the present disclosure are described in detail below. The processes of the present disclosure can be described according to Scheme 1.

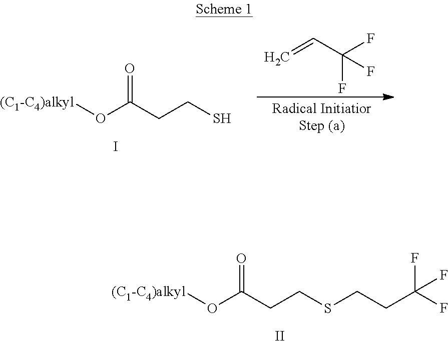

In Step (a) of Scheme 1, a compound of the formula I can be reacted with 3,3,3-trifluoropropene and a radical initiator to provide a compound of the formula II. Step (a) can be performed in the absence of a solvent. It can be advantageous to perform Step (a) in the presence of a solvent.
The radical initiator can be a two-component radical initiator comprising one or more substituted anilines of the formula III

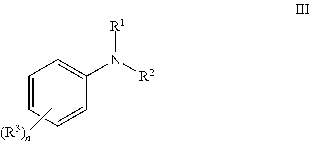

wherein R¹ and R² are each independently H or C₁-C₆ alkyl, wherein each hydrogen in C₁-C₆ alkyl is independently optionally substituted with Cl, Br, I, C₁-C₆ alkyl, or hydroxy; each R³ is independently C₁-C₆ alkyl, —NR⁴R⁵ or —NR⁴C(O)R⁵, wherein each R⁴ and R⁵ is independently H or C₁-C₆ alkyl, and n is an integer from 0 to 3; and a peroxide reagent of the formula IV or formula V

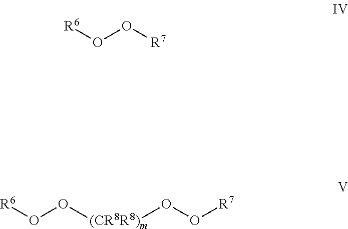

wherein R⁶ and R⁷ are each independently C(O)R⁹, C(R⁹)₃ or H, wherein each R⁸ and R⁹ is independently selected from the group consisting of H, C₁-C₆ alkyl, and C₆-C₁₀ aryl, provided that one of R⁶ or R⁷ is not H, and m is an integer from 2 to 4. In some embodiments, the peroxide reagent can be

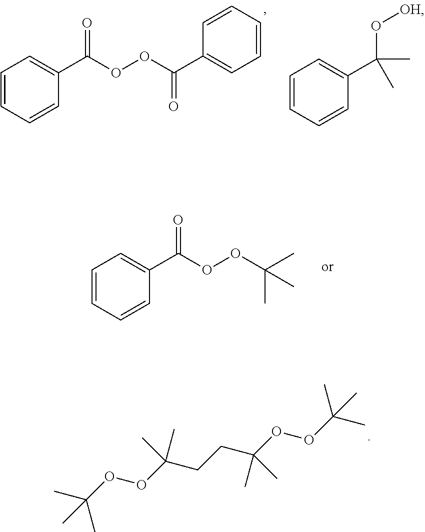

The reaction in Step (a) can be carried out using equimolar 3,3,3-trifluoropropene. It can be advantageous to carry out the reaction of Step (a) using 3,3,3-trifluoropropene in a molar excess compared to the compound of formula I. In some embodiments, 3,3,3-trifluoropropene can be used in about 1 to about 1.5 molar equivalents.

It can be advantageous to carry out the reaction of Step (a) using the compound of formula III in sub-molar equivalents to the compound of formula I. In some embodiments, the compound of formula III can be used in about 0.001 to about 0.2 molar equivalents. In some embodiments, the compound of formula III can be used in about 0.01 to about 0.2 molar equivalents. In some embodiments, the compound of formula III can be used in about 0.05 to about 0.2 molar equivalents.

It can be advantageous to carry out the reaction of Step (a) using the peroxide reagent of formula IV or formula V in sub-molar equivalents to the compound of formula I. In some embodiments, the peroxide reagent of formula IV or formula V can be used in about 0.001 to about 0.2 molar equivalents. In some embodiments, the peroxide reagent of formula IV or formula V can be used in about 0.01 to about 0.2 molar equivalents. In some embodiments, the peroxide reagent of formula IV or formula V can be used in about 0.05 to about 0.2 molar equivalents.

The process of Step (a) can be carried out in a solvent such as acetonitrile (CH₃CN), dioxane, N,N-dimethylformamide (DMF), dichloromethane (DCM), ethyl acetate (EtOAc), N-methyl-2-pyrrolidone (NMP), tetrahydrofuran (THF), toluene, mixtures thereof, and any suitable alternative. In some embodiments, the solvent is toluene, EtOAc, or a mixture thereof. It can be advantageous to carry out the reaction of Step (a) at temperatures of about −20° C. to about 40° C. In some embodiments, the reaction is carried out at a temperature of about −20° C. to about 22° C. In some embodiments, the reaction is carried out at a temperature of about −15° C. to about 10° C. In some embodiments, the process of Step (a) can be carried out for a period of time of about 2 hours to about 24 hours.

The process of Step (a) can be carried out by charging a vessel with the compound of formula I, 3,3,3-trifluoropropene, the peroxide reagent of formula IV or formula V, and the solvent. The compound of formula III can then be added to the reaction vessel. In some embodiments, the process of Step (a) can be carried out by charging a vessel with the compound of formula I, 3,3,3-trifluoropropene, the compound of formula III, and the solvent. The peroxide reagent of formula IV or formula V can then be added to the reaction vessel. In some embodiments, the process of Step (a) can be carried out by charging a vessel with the compound of formula I, the compound of formula III, and the solvent. The peroxide reagent of formula IV or formula V and 3,3,3-trifluoropropene can then be added to the reaction vessel.

In some embodiments, n is 0 and compound III is an N,N-disubstituted aniline. In some embodiments, n is 1 and compound III is a para-methyl N,N-disubstituted aniline. In some embodiment's n is 1 and compound III is a meta-amino N,N-disubstituted aniline. In some embodiments, n is 2 and compound III is a 3,5 dimethyl N,N-disubstituted aniline. In some embodiments, the compound of formula III is selected from the group consisting of

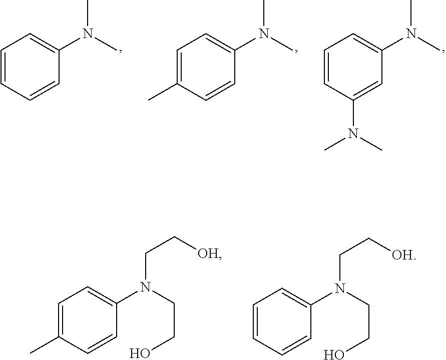

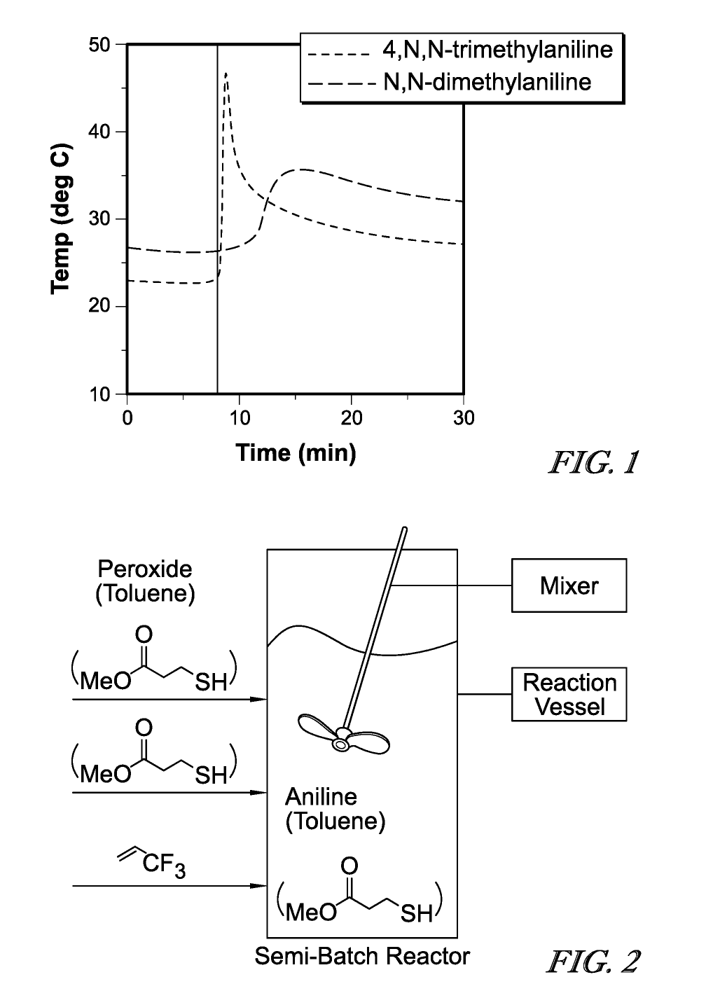

As shown in FIG. 1, the temperature profile of the reaction can be dependent on the compound of formula III used in the reaction. Illustratively, the spike in temperature can be indicative of radical formation.

It was surprisingly discovered that using about 1 mol percent to about 10 mol percent benzoyl peroxide and about 1 mol percent to about 10 mol percent N,N-disubstituted aniline of the formula III, at temperatures of about −15° C. to about 22° C., for a period of time of about 2 hours to about 24 hours, provides a selectivity of desired linear isomer II to undesired branched isomer II-branched of 56:1 or greater.

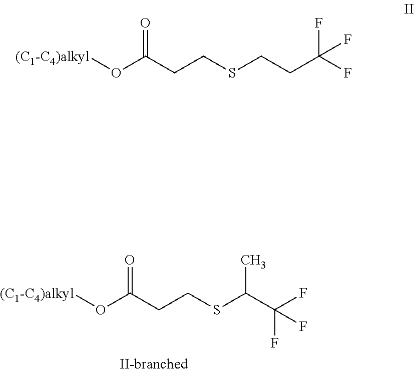

It was also surprisingly discovered that using about 5 mol percent benzoyl peroxide and about 5 mol percent N-phenyldiethanolamine, at temperatures of about −15° C. to about 22° C., for a period of time of about 24 hours, provides a selectivity of the desired linear isomer II to the undesired branched isomer II-branched of about 56:1.

It was more surprisingly discovered that using about 5 mol percent benzoyl peroxide and about 5 mol percent N,N-dimethylaniline, at temperatures of about −15° C. for about 2.5 hours, provides a selectivity of desired linear isomer II to undesired branched isomer II-branched of greater than 95:1.

In an additional embodiment, the disclosure provides a process as shown in Scheme 2.

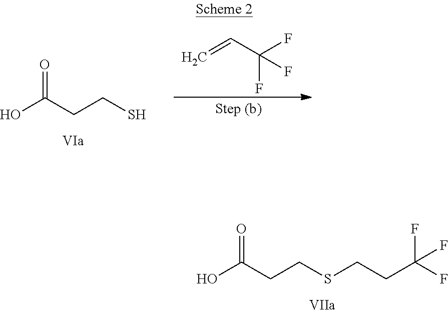

Alternatively, in Scheme 2, 3-((3,3,3-trifluoro-propyl)thio)propanoic acid (VIIa) is prepared by the low temperature free-radical initiated coupling of 3-mercaptopropionic acid (VIa) and 3,3,3-trifluoropropene in the presence of the two-component radical initiator in accordance with the present disclosure. It can be advantageous to carry out the reaction of Step (b) with about 1 equivalent to about 1.5 equivalents of 3,3,3-trifluoropropene. It can be advantageous to carry out the reaction of Step (b) using the peroxide reagent of formula IV or formula V in sub-molar equivalents to the compound of formula VIa. In some embodiments, the benzoyl peroxide can be used at about 0.05 mol percent to about 20 mol percent.

It can be advantageous to carry out the reaction of Step (b) using the compound of formula III in sub-molar equivalents to the compound of formula VIa. In some embodiments, the compound of the formula III can be used at about 0.1 mol percent to about 20 mol percent. In some embodiments, the compound of the formula III is an N,N-dimethylaniline, optionally substituted with one or more OH groups. In some embodiments, the compound of the formula III is N-phenyldiethanolamine.

It can be advantageous to carry out the reaction of Step (b) in the presence of a solvent such as toluene or ethyl acetate. In some embodiments, a solvent is not used. It can be advantageous to carry out the reaction of Step (b) at temperatures of about −20° C. to about 40° C. In some embodiments, the reaction is carried out at a temperature of about −5° C. to about less than 20° C. In some embodiments, the reaction is carried out at a temperature of about 0° C. to about 10° C. The process may be conducted for a period of time of about 2 hours to about 20 hours.

The compound of formula II can undergo further transformations as described according to Scheme 3.

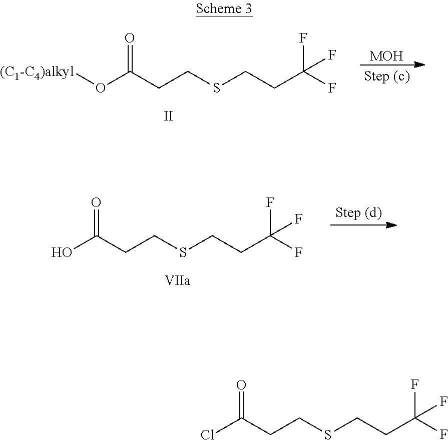

In Step (c) of Scheme 3, the compound of formula II is saponified with a metal hydroxide of the formula MOH, wherein M is selected from the group consisting of sodium (Na), lithium (Li) and potassium (K), to yield 3-((3,3,3-trifluoro-propyl)thio)propanoic acid (VIIa). Step (c) can be performed in the absence of a solvent. It can be advantageous to carry out the reaction of Step (c) in the presence of a polar solvent, such as methanol, tetrahydrofuran or toluene. It can be advantageous to carry out the reaction of Step (c) at temperatures of about 20° C. to about 90° C.

In Step (d) of Scheme 3, 3-((3,3,3-trifluoro-propyl)thio)propanoic acid is chlorinated with thionyl chloride to yield 3-((3,3,3-trifluoropropyl)thio)propanoyl chloride. Step (d) can be performed in the absence of a solvent. It can be advantageous to carry out the reaction of Step (d) in the presence of an aprotic solvent such as ethyl acetate, dichloromethane or toluene. It can be advantageous to carry out the reaction of Step (d) using at a temperature of about 20° C. to about 110° C. to yield 3-((3,3,3-trifluoropropyl)thio)propanoyl chloride. Additional details of Step (d) can be found in U.S. Pat. No. 9,102,654, incorporated herein by reference regarding the preparation of 3-((3,3,3-trifluoropropyl)thio)propanoyl chloride.

The 3-((3,3,3-trifluoropropyl)thio)propanoyl chloride provided by Scheme 3 can be reacted with a 3-chloro-N-ethyl-1-(pyridin-3-yl)-1H-pyrazol-amine as described according to Scheme 4.

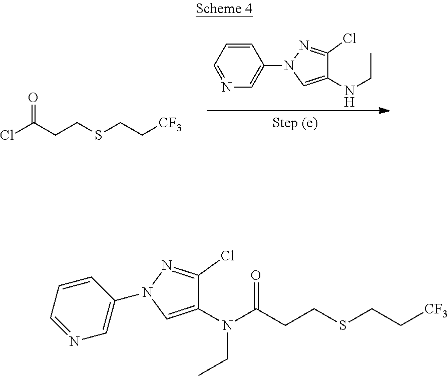 [SMILES: CCNc1cn(-c2cccnc2)nc1Cl ; O=C(Cl)CCSCCC(F)(F)F ; CCN(C(=O)CCSCCC(F)(F)F)c1cn(-c2cccnc2)nc1Cl]

In Step (e) of Scheme 4, 3-((3,3,3-trifluoropropyl)thio)propanoyl chloride can be reacted with 3-chloro-N-ethyl-1-(pyridin-3-yl)-1H-pyrazol-amine in the presence of a base and a solvent to provide the pesticidal thioether N-(3-chloro-1-(pyridin-3-yl)-1H-pyrazol-4-yl)-N-ethyl-3-((3,3,3-trifluoropropyl)thio)-propanamide. Additional details of Step (e) can be found in U.S. Pat. No. 9,102,654, incorporated herein by reference regarding the preparation of the pesticidal thioether N-(3-chloro-1-(pyridin-3-yl)-1H-pyrazol-4-yl)-N-ethyl-3-((3,3,3-trifluoropropyl)thio)-propanamide from 3-((3,3,3-trifluoropropyl)thio)propanoyl chloride.

The reaction of Scheme 1 can be performed in a batch process, a semi-batch process or a continuous process such as a continuous loop reactor process. It should be understood that each process described herein can be optimized to enhance the ratio of compound II to compound II-branched. It should be further understood that the reagent equivalents described herein and reaction conditions can change depending on the chosen process.

The batch process can be carried out by charging the compound of formula I, 3,3,3-trifluoropropene, the peroxide reagent of formula IV or formula V, and the solvent into the reactor. The compound of formula III can then be added. As described herein, operating at lower temperatures can improve the ratio of compound II to compound II-branched by using low-temperature initiators. However, it was surprisingly discovered that the reaction could increase in temperature up to 100° C. under adiabatic conditions, which could result in elevated levels of the branched product II-branched. The batch process provided compound II in good yield with the selectivity of the linear isomer II to the branched isomer II-branched around 90%.

The semi-batch process can be carried out as shown illustratively in FIG. 2. A semi-batch reactor can include a reaction vessel and a mixer as shown in FIG. 2. The semi-batch process can control the rate of reaction by limiting at least one reagent (e.g. 3,3,3-trifluoropropene or the peroxide reagent of formula IV or formula V) and slowly adding over a longer period to spread out heat generation over time.

In some embodiments, the reaction vessel is charged with a compound of the formula III in a solvent as shown in FIG. 2. A first feedstream comprising a peroxide reagent according to formula IV or formula V can be added to the reaction vessel over a period of time. In some embodiments, the first feedstream further comprises a compound of formula I. The feedstream is combined via mixing with the compound of formula III in the reaction vessel. A second feedstream including 3,3,3-trifluoropropene can be added to the reaction vessel over a period of time and combined via mixing with the compound of formula III. In some embodiments, the compound of formula I is included in the reaction vessel prior to addition of the first feedstream or the second feedstream. In some embodiments, the compound of formula I is included in the first feedstream. In some embodiments, the semi-batch process includes a third feedstream comprising the compound of formula I. In some embodiments, each of the first feedstream, the second feedstream, and the third feedstream can be incrementally introduced into the reaction vessel over a series of shots.

Each of the feedstreams can be added to the reaction vessel over different time ranges. The time range for the addition of each feedstream can be one of the following values: about 5 minutes, about 15 minutes, about 30 minutes, about 45 minutes, about 60 minutes, about 75 minutes, about 90 minutes, about 105 minutes, about 120 minutes, about 135 minutes, about 150 minutes, about 165 minutes, about 180 minutes, about 195 minutes, about 210 minutes, about 225 minutes, and about 240 minutes. It is within the scope of the present disclosure for the addition time of each feedstream to fall within one of many different ranges. In a first set of ranges, the addition time of each feedstream can be about 5 minutes to about 240 minutes, about 15 minutes to about 195 minutes, about 15 minutes to about 180 minutes, about 30 minutes to about 180 minutes, about 60 minutes to about 180 minutes, about 90 minutes to about 180 minutes, and about 120 minutes to about 180 minutes. In a second set of ranges, the addition time of each feedstream can be about 15 minutes to about 120 minutes, about 15 minutes to about 90 minutes, and about 15 minutes to about 60 minutes.

In some embodiments, each feedstream can be added in increments, sometimes called shots, to the reaction vessel. Each feedstream (or shot) can be added as a predetermined percentage of the overall amount of the feedstream being added to the reaction vessel. The percentage of the feedstream (or shot) for each increment can be any value, such as about 5%, about 10%, about 15%, about 20%, about 25%, about 30%, and about 35% of the total amount of each feedstream. In some embodiments, the shots are added over the addition time as described above.

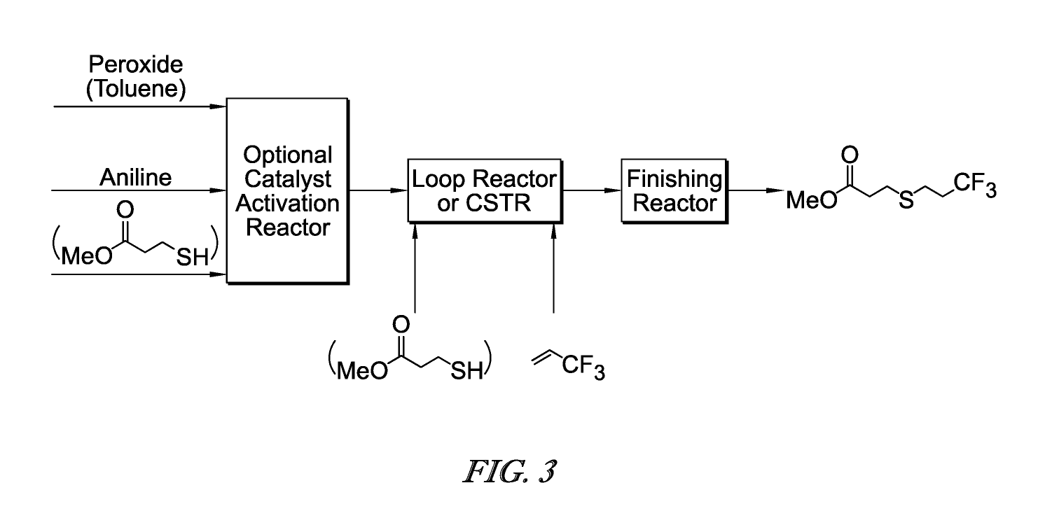

The continuous loop reactor process can be carried out as shown illustratively in FIG. 3. The continuous process could be operated in a single reactor as suggested in FIG. 3 or in several separate reactors. Without being bound by theory, it is it believed that the loop reactor controls the temperature by using a heat exchanger loop with a high recycle ratio to keep the amount of reaction in one pass low so the heat transfer from the heat exchanger can handle the heat generation.

As shown in FIG. 3, the continuous loop process can include an optional catalyst activation reactor. Illustratively, the compound of formula III and/or the peroxide reagent of formula IV or formula V could be pre-activated in the catalyst activation reactor prior to entering the loop reactor. In some embodiments, the continuous loop reactor process can include a finishing reactor after the loop reactor. In some embodiments, the loop reactor could also be replaced with a continuously stirred tank reactor (CSTR). In some embodiments, the catalyst activation reactor and the finishing reactor can be loops, CSTRs, tube, or other single liquid phase continuous reactors.

In some embodiments, the compound of formula III and the peroxide reagent of formula IV or formula V are pre-mixed in an optional catalyst activation reactor. After a set mixing time, a first feedstream mixture can flow from the catalyst activation reactor to the loop reactor and interact with a second feedstream comprising 3,3,3-trifluoropropene. In some embodiments, the compound of formula I is added in the catalyst activation reactor. In some embodiments, the compound of formula I is added in the loop reactor.

In some embodiments, the continuous loop process does not have a catalyst activations reactor. In some embodiments, the compound of formula III is in a first feedstream and the peroxide reagent of formula IV or formula V is in a second feedstream. In some embodiments, the 3,3,3-trifluoropropene is in a third feedstream. In some embodiments, the compound of formula I is in a fourth feedstream. In some embodiments, the compound of formula I is added in the loop reactor.

The loop reactor can be cooled by flowing a cooling liquid through the reactor. It can be advantageous to apply the cooling liquid to an exterior of the heat exchanger section. In some embodiments, the cooling liquid has a temperature of about 5° C. In some embodiments, each of the first feedstream and second feedstream can be cooled prior to entering the loop reactor. After mixing in the loop reactor, the reaction mixture can then flow from the loop reactor to the finishing reactor to allow the reaction to progress and provide the compound of formula II.

In some embodiments, the continuous loop reactor process comprises a first feedstream including 3,3,3-trifluoropropene, a second feedstream including a compound of the formula III, a third feedstream including a compound of the formula IV or the formula V, and a fourth feedstream including a compound of the formula I. In some embodiments, each of the feedstreams includes a solvent. It can be advantageous that only some of the feedstreams include a solvent. In some embodiments, the solvent is toluene.

Both the continuous loop reactor process and the semi-batch reactor process can achieve higher yield by controlling the radical concentration. The continuous loop reactor process operates at high conversion with a feedstream of initiators maintaining radicals in the loop reactor. It can be advantageous in a semi-batch process as described herein to provide a continuous feedstream of the peroxide reagent of formula IV or formula V into the reaction vessel to generate radicals as the reagents are added. It can be advantageous in the semi-batch process for the continuous feedstream of the peroxide reagent of formula IV or formula V to include 3,3,3-trifluoropropene.

The processes described herein can further include a purification step to isolate the linear compound II. In some embodiments, the processes described herein may provide compounds of the formulae II and II-branched that may need to be separated. Due to similarities in molecular structure, purification via extractive techniques or column chromatography may not be the most efficient method of purification. Described herein is a purification process using a multistage fractional distillation.

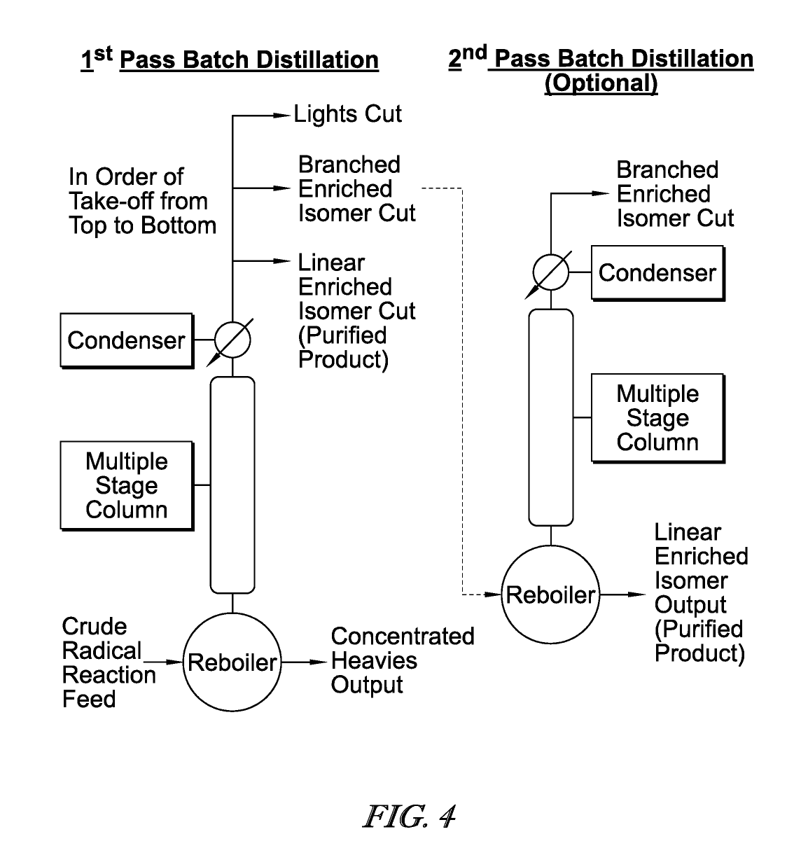

The multistage fractional distillation in accordance with the present disclosure is shown, for example, in FIG. 4. A first distillation apparatus can include a reboiler and a column. The reboiler is charged with the crude reaction mixture, as shown in FIG. 4, and heated. The vapor from the reboiler enters a multiple stage column such as an Oldershaw column or a Goodloe packed column, although any suitable fractional distillation column can be used. A vacuum can be applied to the system to minimize the temperature of the reboiler. In some embodiments, the multiple stage columns can be fitted with a condenser and/or a valve splitter.

The reboiler temperature can be selected from a range of temperatures. The reboiler temperature can be at a temperature of about 50° C., about 60° C., about 70° C., about 80° C., about 90° C., about 95° C., about 100° C., about 105° C., about 110° C., about 115° C., about 120° C., about 125° C., about 130° C., about 135° C., about 140° C., about 145° C., about 150° C., about 155° C., about 160° C., or about 170° C. It is within of the scope present disclosure for the reboiler temperature to fall within one of many different ranges. In some embodiments, the reboiler temperature can be about 50° C. to about 170° C., about 80° C. to about 155° C., about 90° C. to about 155° C., about 90° C. to about 150° C., about 90° C. to about 140° C., about 100° C. to about 140° C., about 110° C. to about 140° C., or about 110° C. to about 130° C.

The vacuum pressure can be selected from a range of pressures. The vacuum pressure can be about 1 torr, about 5 torr, about 10 torr, about 15 torr, about 20 torr, about 25 torr, about 30 torr, about 35 torr, about 40 torr, about 45 torr, about 50 torr, about 75 torr, about 100 torr, about 150 torr, about 200 torr, about 250 torr, about 300 torr, about 350 torr, about 400 torr, about 450 torr, about 500 torr, about 550 torr, about 600 torr, about 650 torr, about 700 torr, about 750 torr, or about 760 torr. It is within of the scope present disclosure for the vacuum pressure to fall within one of many different ranges, such as about 1 torr to about 760 torr, about 1 torr to about 500 torr, about 1 torr to about 300 torr, about 1 torr to about 10 torr, about 5 torr to about 50 torr, 5 torr to about 40 torr, about 5 torr to about 30 torr, about 5 torr to about 20 torr, or about 5 torr to about 15 torr.

The multistage fractional distillation process can be carried out using a reflux ratio, sometimes called a reflux to take-off ratio of from about 1:1 to about 10:1. In some embodiments, the reflux ratio is about 3:1. In some embodiments, the reflux ratio is about 5:1. In some embodiments, the reflux ratio is about 10:1. In one aspect, the ratios are determined by optimizing the separation power of the column, the feed composition, and the heat balance.

In some embodiments, fractions can be isolated from the distillation. Illustratively, the fractions are recovered overhead following the reflux splitter. Illustratively, the first fractions can include solvents and unreacted compound of the formula I. The subsequent fractions can be enriched in the branched product II-branched followed by linear enriched product II. The separation efficiency and recovery can be impacted by the number of equilibrium stages present in the distillation column, as well as how the column is operated at a reflux ratio and the extent of which the distillation is completed. In one aspect, the fractions are decided based on changes in the boiling point of the bottoms as a function of the applied vacuum.

In an example, the compound of formula I is collected when the reboiler temperature is between 90° C. and 150° C. and the vacuum is about 50 torr. In some embodiments, as the vacuum is increased from 50 torr to between about 5 torr to about 15 torr the branched isomer is removed. In some embodiments, the linear isomer is removed when the vacuum is in the range of about 5 torr to about 15 torr and the reboiler is in a range of about 90° C. to about 150° C.

In some embodiments, the fractions from the first distillation apparatus can be combined and further purified by a second distillation apparatus as shown in FIG. 4. The second distillation apparatus is similar to the first distillation apparatus. During this second distillation, the linear product II remains in the reboiler while the branched product II-branched is removed as a column fraction. The second pass distillation of the branched-enriched isomer II-branched cuts can further increase the recovery and purity of the linear isomer to approximately 98% each. The reboiler and vacuum conditions here are similar to those described above and are incorporated. In an example, the reboiler temperature is in a range of about 110° C. to about 140° C. and the vacuum pressure is in a range of about 5 torr to about 10 torr. In one aspect, the fractions are decided based on changes in the boiling point of the bottoms as a function of the applied vacuum.

In some embodiments, the present disclosure provides a process for preparing a compound of the formula II

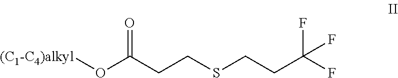

comprising

contacting a compound of the formula I

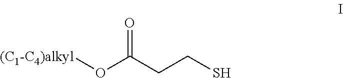

with 3,3,3-trifluoropropene, in the presence of a compound of formula III

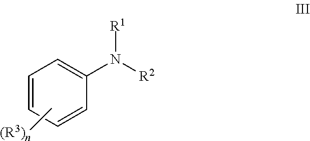

wherein R¹ and R² are each independently H or C₁-C₆ alkyl, wherein each hydrogen in C₁-C₆ alkyl is independently optionally substituted with Cl, Br, I, C₁-C₆ alkyl, or hydroxy; each R³ is independently C₁-C₆ alkyl, —NR⁴R⁵ or —NR⁴C(O)R⁵, wherein each R⁴ and R⁵ is independently H or C₁-C₆ alkyl, and n is an integer from 0 to 3; and a peroxide reagent of the formula IV or formula V

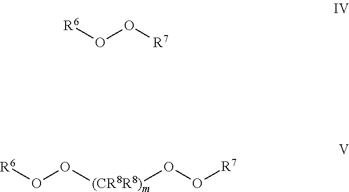

wherein R⁶ and R⁷ are each independently C(O)R⁹, C(R⁹)₃ or H, wherein each R⁸ and R⁹ is independently selected from the group consisting of H, C₁-C₆ alkyl, and C₆-C₁₀ aryl, provided that one of R⁶ or R⁷ is not H, and m is an integer from 2 to 4.

### CHEMISTRY EXAMPLES

### Materials and Methods

These examples are for illustration purposes and are not to be construed as limiting this disclosure to only the embodiments disclosed in these examples.

Starting materials, reagents, and solvents that were obtained from commercial sources were used without further purification. Melting points are uncorrected. Examples using “room temperature” were conducted in climate controlled laboratories with temperatures ranging from about 20° C. to about 24° C. Molecules are given their known names, named according to naming programs within Accelrys Draw, ChemDraw, or ACD Name Pro. If such programs are unable to name a molecule, such molecule is named using conventional naming rules. ¹H NMR spectral data are in ppm (δ) and were recorded at 300, 400, 500, or 600 MHz; ¹³C NMR spectral data are in ppm (δ) and were recorded at 75, 100, or 150 MHz, and ¹⁹F NMR spectral data are in ppm (δ) and were recorded at 376 MHz, unless otherwise stated.

### Example 1—Preparation of Compound N-(3-chloro-1-(pyridin-3-yl)-1H-pyrazol-4-yl)-N-ethyl-3-((3,3,3-trifluoropropyl)thio)propanamide from methyl 3-mercaptopropionate

### Step 1. Preparation of methyl 3-((3,3,3,-trifluoropropyl)thio)propionate

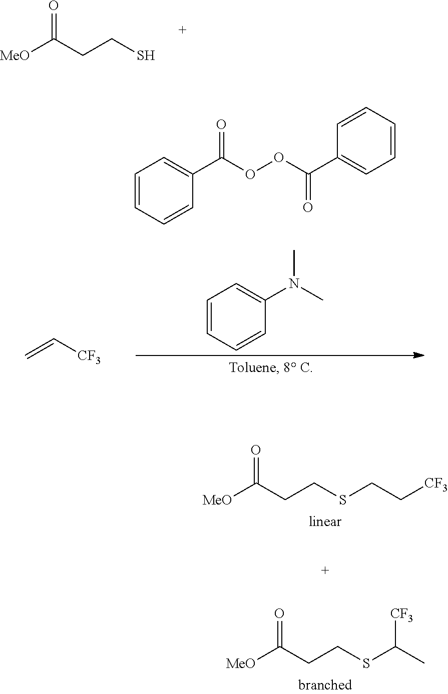

A 100 mL stainless steel Parr reactor was charged with methyl 3-mercaptopropionate (15.04 g, 125.2 mmol), toluene (25.3 g), and benzoyl peroxide (Luperox A75, 2.09 g, 6.47 mmol), and the reactor was purged with nitrogen and pressure checked. The reactor was cooled to 8° C. with an ice bath and 3,3,3-trifluoropropene (12.70 g, 132.2 mmol) was added via transfer cylinder. N,N-Dimethylaniline (0.7200 g, 5.900 mmol) was added with a pressure rated syringe, and the reaction was stirred at 6-8° C. for 3 hours. The crude mixture was quantified (62.9 g, 36.9 wt. %, 23.2 g active, 86%, 56:1 linear:branched by GC).

Sodium hydroxide (10%, 12.8 g) was added to the toluene solution and was stirred at 17° C. for 40 minutes. The layers were allowed to separate, and the aqueous layer was removed. Hydrochloric acid (2 N, ˜10 mL) was added to the toluene layer and was mixed at 17° C. for 15 minutes. The aqueous layer was removed and after rinsing with toluene (7.2 g). The organic layer was isolated and quantified (63.28 g, 35.8 wt. %, 22.7 g active, 84%). Linear to branched selectivity was determined by analysis of the crude mixture (56:1 linear:branched by GC): ¹H NMR (400 MHz, CDCl₃) δ 3.71 (s, 3H), 2.82, (td, J=7.3, 0.7 Hz, 2H), 2.75-2.68 (m, 2H), 2.63 (td, J=7.2, 0.6 Hz, 2H), 2.47-2.31 (m, 2H); ¹³C NMR (101 MHz, CDCl₃) δ 172.04, 125.93 (q, J=277.2 Hz), 51.86, 34.68 (q, J=28.6 Hz), 34.39, 27.06, 24.11 (q, J=3.3 Hz); ¹⁹F NMR (376 MHz, CDCl₃) δ −66.53.

### Alternative Synthesis of methyl 3-((3,3,3,-trifluoropropyl)thio)propionate

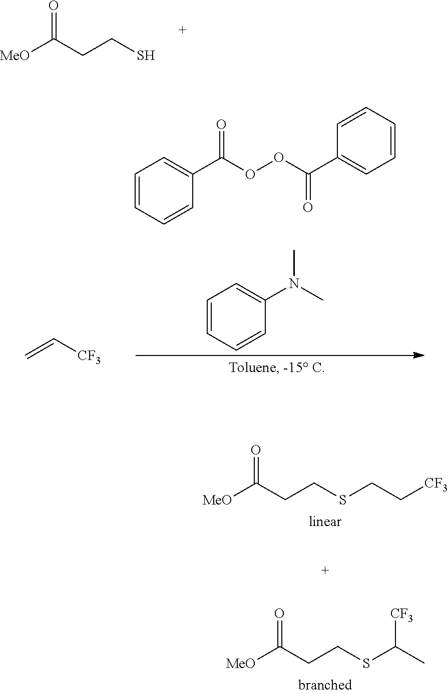

A 100 mL stainless steel Parr reactor was charged with methyl 3-mercaptopropionate (15.3 g, 127 mmol), toluene (25.4 g), and benzoyl peroxide (Luperox A75, 2.04 g, 6.32 mmol). The reactor was purged with nitrogen and pressure checked. The reactor was cooled to −16° C. with an ethanol/ethylene glycol/dry ice bath, and 3,3,3-trifluoropropene (12.4 g, 129 mmol) was added via transfer cylinder. N,N-Dimethylaniline (0.660 g, 5.40 mmol) was added with a pressure rated syringe, and the reaction was stirred at −15° C. for 4 hours. The reaction was allowed to slowly warm to room temperature overnight and the crude mixture was quantified (53.2 g, 42.4 wt %, 22.56 g active, 84%, 95:1 linear:branched by GC).

### Alternative Synthesis of methyl 3-((3,3,3-trifluoropropyl)thio)propanoate

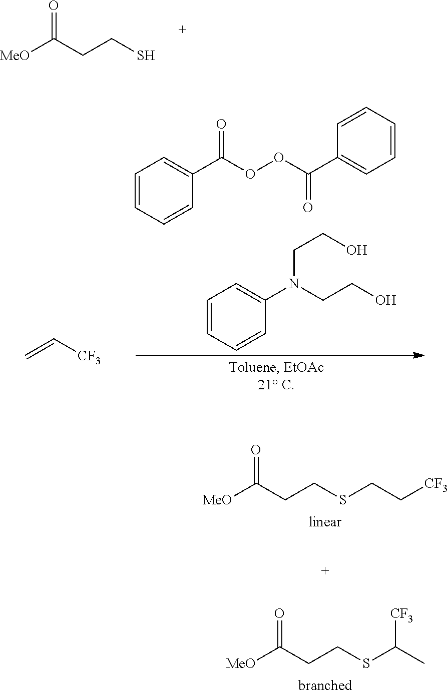

A 100 mL stainless steel Parr reactor was charged with methyl 3-mercaptopropionate (15.2 g, 126 mmol), toluene (25.3 g), and benzoyl peroxide (Luperox A75, 2.07 g, 6.4 mmol). The reactor was purged with nitrogen and pressure checked. 3,3,3-Trifluoropropene (12.3 g, 128 mmol) was added via transfer cylinder. N-phenyldiethanolamine (1.2 g, 6.30 mmol) in ethyl acetate (3.92 g) was added with a pressure rated syringe, and the reaction was stirred at 21° C. for 18 hours. The solution was removed from the reactor and stirred with 17.3 g 1 N sodium hydroxide for 30 minutes and the layers were allowed to separate. 18.9 g of aqueous layer and 61.7 g organic layer were isolated. The organic layer was quantified (61.7 g, 33.4 wt. %, 20.6 h active, 76% yield, 32:1 linear:branched by GC).

### Step 2. Preparation of 3-((3,3,3,-trifluoropropyl)thio)propionate

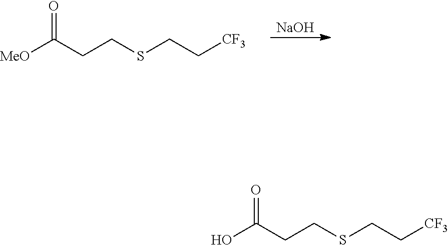

A 1 L jacketed reactor (jacket at 25° C.) equipped with a thermocouple and nitrogen inlet was charged with methyl 3-((3,3,3-trifluoropropyl)thio)propionate (78.80 g, 344.0 mmol). Sodium hydroxide (10 weight percent, 157.8 g, 394.5 mmol) was added making a cloudy mix (biphasic), and the mixture was stirred at room temperature. After 1.5 hours, the mixture became homogeneous. Hydrochloric acid (2 N, 274.3 g, 532.6 mmol) was added forming a biphasic mixture with the product as an oil on the bottom layer. Toluene (435.5 g) was added, and the phases were separated. Some of the toluene was atmospherically distilled to azeotropically remove water. The resulting bottoms toluene solution was quantified (458.0 g, 14.8 wt. %, 67.7 g active, 97%).

### Step 3: Preparation of 3-((3,3,3,-trifluoropropyl)thio)propanoyl chloride

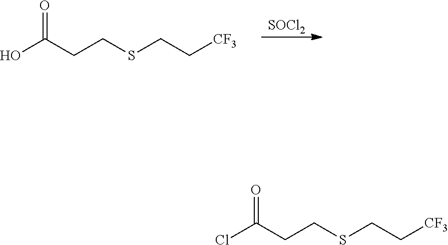

A dry 5 L round bottom flask equipped with magnetic stirrer, nitrogen inlet, reflux condenser, and thermometer, was charged with 3-((3,3,3-trifluoropropyl)thio)propanoic acid (188 g, 883 mmol) in dichloromethane (CH₂Cl₂) (3 L). Thionyl chloride (525 g, 321 mL, 4.42 mol) was then added dropwise over 50 minutes (min). The reaction mixture was heated to reflux (about 36° C.) for two hours (h), and then cooled to room temperature (RT, about 22° C.). Concentration under vacuum on a rotary evaporator, followed by distillation (40 Torr, product collected from 123-127° C.) gave the title compound as a clear colorless liquid (177.3 g, 86%): ¹H NMR (400 MHz, CDCl₃) δ 3.20 (t, J=7.1 Hz, 2H), 2.86 (t, J=7.1 Hz, 2H), 2.78-2.67 (m, 2H), 2.48-2.31 (m, 2H); ¹⁹F NMR (376 MHz, CDCl₃) δ −66.42, −66.43, −66.44, −66.44.

### Step 4. Preparation of N-(3-chloro-1-(pyridin-3-yl)-1H-pyrazol-4-yl)-N-ethyl-3-((3,3,3-trifluoropropyl)thio)propanamide

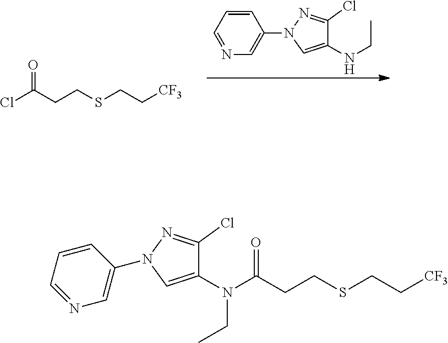 [SMILES: CCNc1cn(-c2cccnc2)nc1Cl ; O=C(Cl)CCSCCC(F)(F)F ; CCN(C(=O)CCSCCC(F)(F)F)c1cn(-c2cccnc2)nc1Cl]

A three-neck round bottomed flask (100 mL) was charged with 3-chloro-N-ethyl-1-(pyridin-3-yl)-1H-pyrazol-amine (5.00 g, 22.5 mmol) and ethyl acetate (50 mL). Sodium bicarbonate (4.72 g, 56.1 mmol) was added, followed by dropwise addition of 3-((3,3,3-trifluoropropyl)thio)propanoyl chloride (5.95 g, 26.9 mmol) at <20° C. for 2 hours, at which point HPLC analysis indicated that the reaction was complete. The reaction was diluted with water (50 mL) (off-gassing) and the layers separated. The aqueous layer was extracted with ethyl acetate (20 mL) and the combined organic layers were concentrated to dryness to afford a light brown solid (10.1 g, quantitative). A small sample of crude product was purified by flash column chromatography using ethyl acetate as eluent to obtain an analytical reference sample: mp 79-81° C.; ¹H NMR (400 MHz, DMSO-d₆) δ 9.11 (d, J=2.7 Hz, 1H), 8.97 (s, 1H), 8.60 (dd, J=4.8, 1.4 Hz, 1H), 8.24 (ddd, J=8.4, 2.8, 1.4 Hz, 1H), 7.60 (ddd, J=8.4, 4.7, 0.8 Hz, 1H), 3.62 (q, J=7.2 Hz, 2H), 2.75 (t, J=7.0 Hz, 2H), 2.66-2.57 (m, 2H), 2.57-2.44 (m, 2H), 2.41 (t, J=7.0 Hz, 2H), 1.08 (t, J=7.1 Hz, 3H); ESIMS m/z 407 ([M+H]+).

### COMPARATIVE EXAMPLES

### Example CE-1 Synthesis of methyl 3-((3,3,3-trifluoropropyl)thio)propionate

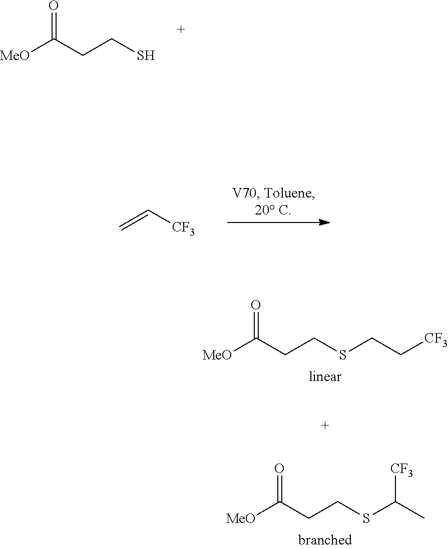

Example CE-1 is a comparative example wherein methyl 3-mercaptopropionate and 3,3,3-trifluoropropene are coupled with the radical initiator, 2,2′-azobis(4-methoxy-2,4-dimethyl) valeronitrile (initiates at temperatures greater than 20° C.). These conditions afforded a selectivity of the desired linear isomer to the undesired branched isomer of about 24:1. In contrast, the two-component radical initiator system, comprising benzoyl peroxide and N-phenyldiethanolamine provided a 140% selectivity improvement over the radical initiator 2,2′-azobis(4-methoxy-2,4-dimethyl) valeronitrile. Furthermore, the two-component radical initiator system, comprising benzoyl peroxide and N,N-dimethylaniline provided a 238% selectivity improvement over the radical initiator 2,2′-azobis(4-methoxy-2,4-dimethyl) valeronitrile.

A 100 mL stainless steel Parr reactor was charged with methyl 3-mercaptopropionate (4.15 g, 34.5 mmol), toluene (30.3 g), and 2,2′-azobis(4-methoxy-2,4-dimethyl) valeronitrile (0.531 g, 1.72 mmol) and the reactor was cooled with a dry ice/acetone bath, purged with nitrogen, and pressure checked. 3,3,3-Trifluoropropene (3.40 g, 35.4 mmol) was added via transfer cylinder and the reaction was allowed to warm to 20° C. After 23 hours, the reaction was heated to 50° C. for 1 hour to decompose any remaining valeronitrile initiator. The reaction was allowed to cool to room temperature. The solution was concentrated to provide the title compound (7.01 g, 66%, 70.3 wt. % linear isomer by GC internal standard assay, 4.93 g active, 66%, 24:1 linear:branched by GC, 18:1 linear:branched by fluorine NMR): ¹H NMR (400 MHz, CDCl₃) δ 3.71 (s, 3H), 2.82, (td, J=7.3, 0.7 Hz, 2H), 2.75-2.68 (m, 2H), 2.63 (td, J=7.2, 0.6 Hz, 2H), 2.47-2.31 (m, 2H); ¹³C NMR (101 MHz, CDCl₃) δ 172.04, 125.93 (q, J=277.2 Hz), 51.86, 34.68 (q, J=28.6 Hz), 34.39, 27.06, 24.11 (q, J=3.3 Hz); ¹⁹F NMR (376 MHz, CDCl₃) δ −66.53.

### Example CE-2 Photochemical Synthesis of methyl 3-((3,3,3-trifluoropropyl)thio)propanoate

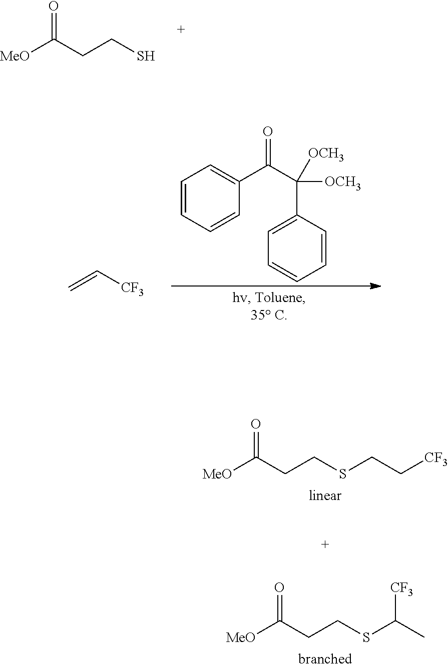

Example CE-2 is a comparative example wherein methyl 3-mercaptopropionate and 3,3,3-trifluoropropene are coupled using photochemical conditions with the radical initiator, 2,2-dimethoxy-2-phenylacetophenone. The reaction was initiated by a UV lamp (366 nm). These conditions afforded a selectivity of the desired linear isomer to the undesired branched isomer of about 21:1. In contrast, the two-component radical initiator system, comprising benzoyl peroxide and N-phenyldiethanolamine provided a 260% selectivity improvement over the radical initiator 2,2-dimethoxy-2-phenylacetophenone. Furthermore, the two-component radical initiator system, comprising benzoyl peroxide and N,N-dimethylaniline provided a 452% selectivity improvement over the radical initiator 2,2-dimethoxy-2-phenylacetophenone.

A 500 mL three-neck round-bottomed flask was charged with toluene (200 mL) and cooled to <−50° C. with a dry ice/acetone bath. 3,3,3-Trifluoropropene (21.8 g, 227 mmol) was condensed into the reaction by bubbling the gas through the cooled solvent and the ice bath was removed. Methyl 3-mercaptopropionate (26.8 g, 223 mmol) and 2,2-dimethoxy-2-phenylacetophenone (2.72 g, 10.61 mmol) were added and a UVP lamp (4 watt) that was placed within 2 centimeters of the glass wall was turned on to the long wave function (366 nanometers). The reaction reached 35° C. due to heat from the lamp. After 4 hours, all of the trifluoropropene was either consumed or boiled out of the reaction. The light was turned off and the reaction stirred at room temperature overnight. After 22 hours, more 3,3,3-trifluoropropene (3.1 g) was bubbled through the mixture at room temperature and the light was turned on for an additional 2 hours. The reaction had converted 93% so no more trifluoropropene was added. The light was turned off and the mixture concentrated on the rotovap (40° C., 20 torr) giving a yellow liquid (45.7 g, 21:1 linear:branched isomer, 75 wt. % pure linear isomer determined by a GC internal standard assay, 34.3 g active, 71% in pot yield). Characterization matched sample prepared by the previous method.

### Example CE-3 Synthesis of methyl 3-((3,3,3-trifluoropropyl)thio)propanoate

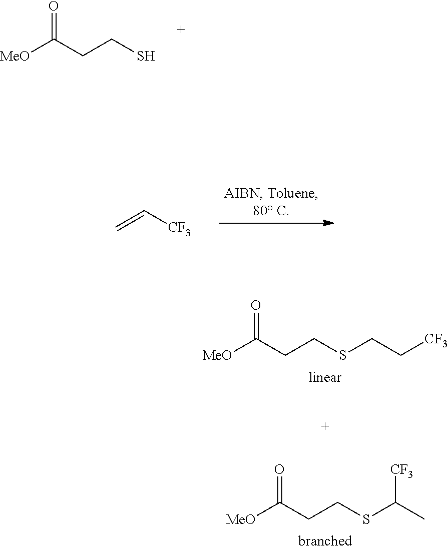

Example CE-3 is a comparative example wherein methyl 3-mercaptopropionate and 3,3,3-trifluoropropene are coupled with the radical initiator α-azobisisobutyronitrile (AIBN, initiates at temperatures greater than 60° C.) at temperatures greater than 60° C. These conditions afforded a selectivity of the desired linear isomer to the undesired branched isomer of about 10:1. In contrast, the two-component radical initiator system, comprising benzoyl peroxide and N-phenyldiethanolamine provided a 560% selectivity improvement over the radical initiator α-azobisisobutyronitrile (AIBN). Furthermore, the two-component radical initiator system, comprising benzoyl peroxide and N,N-dimethylaniline provided a 950% selectivity improvement over the radical initiator α-azobisisobutyronitrile (AIBN).

A 2 L autoclave reactor was charged with toluene (716.45 g), methyl 3-mercaptopropionate (187.78 g, 1562.6 mmol), and AIBN (12.890 g, 78.500 mmol). The reactor was sealed and pressurized with nitrogen (˜100 psig) three times to purge the system of air. 3,3,3-Trifluoropropene (153.20 g, 1595.0 mmol) was added via transfer cylinder at 12° C. (cold water bath). The reaction was heated to 80° C. and stirred for 21 hours. The reaction was allowed to cool to room temperature and vacuum transferred out of the reactor. The crude solution was concentrated by rotary evaporation (bath: 40° C., 12 mm Hg) to providing a clear yellow liquid (371.95 g, 9.8:1 linear:branched isomer, 69 wt. % pure linear isomer determined by a GC internal standard assay, 257.39 g active, 76% in pot yield). Characterization matched sample prepared by the previous method.

### Semi-Batch Process Example

The reactor in this process was a 300 mL stainless steel reactor, which was charged with 50 g of methyl 3-mercaptopropionate and 2.81 g (0.05 mol eq.) of 4,N,N-trimethylaniline. The reactor was assembled and inerted with 80 psig of nitrogen. A 500 mL Isco syringe pump was charged with 45 g (1.05 eq.) of 3,3,3-trifluoropropene and pressurized to >80 psig to liquefy the material. A second Isco syringe pump was filled with 17.1 g of a 12 wt. % solution of benzoyl peroxide in toluene (0.01 eq.). The reactor was cooled to 4° C. with a circulating chiller pumping cooling fluid through a coil wrapped around the vessel. The vessel was mixed at 1000 rpm with a sparging impeller. The two pumps were started simultaneously and delivered material to the reactor at a constant rate over 120 min into the headspace of the reactor. The reactor was allowed to mix for an additional 120 min before warming to room temperature and continuing to mix for an additional 18 h. The reactor was vented and 129.8 g of product solution was recovered containing 66 wt. % product corresponding to 95% yield. The linear:branch ratio was 79:1.

### Continuous Loop Reactor Process Example

The reactor in this process was a 70 mL tube-in-tube reactor. The process fluid flowed on the inside of the reactor and a circulating cooling bath was operated at 5° C. on the shell side of the reactor to control temperature. Each feedstream was precooled prior to entering the loop reactor. The reactor pressure was maintained at 100 psig with a backpressure controller on the loop outlet line. The reactor pump-around loop was run with a centrifugal pump operating at >5,000 mL/min. Samples were collected from the product line prior to accumulating in a product tank. A 500 mL Isco syringe pump was filled with 3,3,3-trifluoropropene and pressurized to >70 psig to liquefy the gas and delivered to the reactor at 1.42 mL/min. A second 500 mL Isco syringe pump was filled with 22 wt. % 4,N,N-trimethylaniline dissolved in toluene and fed to the reactor at 0.46 mL/min. A solution of 9.5 wt. % benzoyl peroxide dissolved in toluene was made up and pumped at 2.02 mL/min using an HPLC dual piston pump. A fourth feed of 57 wt. % methyl 3-mercaptopropionate was also fed to the reactor with an HPLC dual piston pump at 2.02 mL/min. These conditions correspond to molar equivalents to methyl 3-mercaptopropionate of 1.05 for 3,3,3-trifluoropropene and 0.05 for 4,N,N-trimethylaniline and benzoyl peroxide. The mean residence time in the reactor was 10 min. The outlet flow rate was 6.4 g/min at a product concentration of 34.5 wt. %. This corresponds to 76% crude yield. The linear:branch ratio was 43:1.

### PURIFICATION EXAMPLES

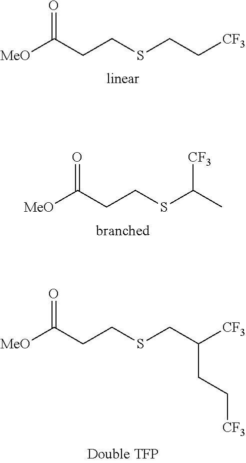 [SMILES: COC(=O)CCSCCC(F)(F)F ; COC(=O)CCSC(C)C(F)(F)F ; COC(=O)CCSCC(CCC(F)(F)F)C(F)(F)F]

### Example 1

The crude methyl ester side chain products (e.g. linear, branched, double TFP), post rotovap concentration (758.4 g crude, 69.4 wt. % linear methyl ester) solution was charged to the 2 L still equipped with a 1″ diameter, 10 tray vacuum jacketed Oldershaw column equipped with a condenser and a tic-toc valve flow splitter. After initial distillation of the lights at 15 mmHg, the distillation was completed at 7-8 mmHg vacuum. Under these conditions, the branched isomer and the linear methyl ester isomer were distilled over between 78.8° C. and 89.4° C. for the overheads temperature and 112.0° C. to 126.9° C. for the bottoms temperature. The distillation was carried out until only a small amount of visible liquid remained in the bottom of the still—the level was below the top of the magnetic stir bar. Periodically during the distillation, the overheads were collected, with 9 overall collections or cuts being taken in this experiment. For more detail about the size of each cut, and the quantification of the compounds in the overhead cuts by GC, see Table 1.

TABLE 1
Recovery of linear isomer as a function of cut combinations.

| | | | Wt. % | Wt. % | |
|---|---|---|---|---|---|
| | Physical | Grams | Linear | Branched) | Recovery |
| Description | Grams | Linear | (%) | (%) | Linear (%) |
| Starting Amount | — | 526 | — | — | 100.0% |
| (100%) | | | | | |
| Combine Cuts 2-9 | 544 | 481 | 88.4 | 8.9% | 91.4% |
| Combine Cuts 3-9 | 537 | 480 | 89.4 | 8.8% | 91.2% |
| Combine Cuts 4-9 | 506 | 458 | 90.6 | 7.9% | 87.1% |
| Combine Cuts 5-9 | 454 | 418 | 92.1 | 6.7% | 79.4% |
| Combine Cuts 6-9 | 408 | 379 | 93.0 | 5.8% | 72.1% |
| Combine Cuts 7-9 | 322 | 307 | 95.6 | 3.4% | 58.4% |
| Combine Cuts 8-9 | 241 | 234 | 97.1 | 1.8% | 44.4% |
| Combine Cuts 9-9 | 165 | 162 | 98.2 | 0.8% | 30.6% |
| Residual Linear in | — | 40 | — | — | 7.5% |
| bottoms layer | | | | | |

| | |
|---|---|
| Total Recovered | 521 |
| Cuts + Bottom | |
| % Closure′ | 99.0% |

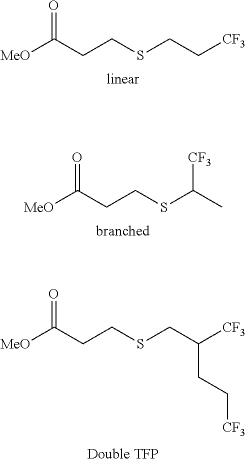 [SMILES: COC(=O)CCSCCC(F)(F)F ; COC(=O)CCSC(C)C(F)(F)F ; COC(=O)CCSCC(CCC(F)(F)F)C(F)(F)F]

### Example 2

The crude methyl ester side chain products (e.g. linear, branched, double TFP), post rotovap concentration (721.9 g crude, 68.9 wt. % linear methyl ester) solution was charged to the 2 L still equipped with a 1″ diameter, 24 tray vacuum jacketed Oldershaw column equipped with a condenser and a tic-toc valve flow splitter, as shown in FIG. 4. The distillation was completed at 7-8 mmHg vacuum. Under these conditions, the bulk of the branched and linear methyl ester isomers were distilled over between 68.9° C. and 93.7° C. for the overheads temperature and 124.1° C. to 156.4° C. for the bottoms temperature. The distillation was carried out until only a small amount of visible liquid remained in the bottom of the still—the level was below the top of the magnetic stir bar. Periodically during the distillation, the overheads were collected, with 6 overall collections or cuts being taken in this experiment. For more detail about the size of each cut, and the quantification of the compounds in the overhead cuts by GC, see Table 2.

TABLE 2
Recovery of linear isomer as a function of cut combinations

| | | | Wt. % | Wt. % | |
|---|---|---|---|---|---|
| | Physical | Grams | Linear | Branched | Recovery |
| Description | Grams | Linear | (%) | (%) | Linear (%) |
| Starting Amount | | 497 | | | 100.0 |
| (100%) | | | | | |
| Combine Cuts 1-6 | 506 | 448 | 88.5 | 8.8 | 90.1 |
| Combine Cuts 2-6 | 497 | 448 | 90.0 | 8.7 | 90.0 |
| Combine Cuts 3-6 | 472 | 440 | 93.1 | 5.8 | 88.5 |
| Combine Cuts 4-6 | 443 | 423 | 95.6 | 3.6 | 85.1 |
| Combine Cuts 5-6 | 395 | 388 | 98.0 | 1.3 | 77.9 |
| Combine Cuts 6-6 | 352 | 348 | 99.1 | 0.4 | 70.0 |
| Residual Linear in | | 34 | | | 6.8 |
| bottoms layer | | | | | |

| | |
|---|---|
| Total Recovered | 482 |
| Cuts + Bottom | |
| % Closure | 96.9% |

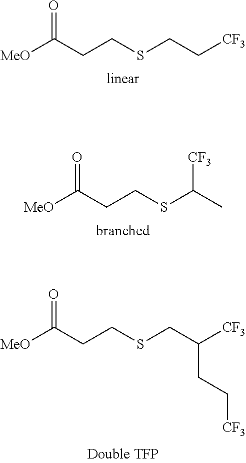 [SMILES: COC(=O)CCSCCC(F)(F)F ; COC(=O)CCSC(C)C(F)(F)F ; COC(=O)CCSCC(CCC(F)(F)F)C(F)(F)F]

### Example 3

The crude methyl ester side chain products (e.g. linear, branched, double TFP), post toluene stripping distillation (31.2 kg crude, 85.6 wt. % linear methyl ester) solution was charged to the 10 gallon still equipped with a 4″ diameter, 5′ tall packed column, with GOODLOE high-efficiency packing, and a condenser and a valve capable of reflux/take-off splitting. The distillation was completed at around 5 mm Hg vacuum at the top of the column. The reflux ratio employed during this distillation was variable, but the bulk of the product take-off cuts (5-12) were taken with a 5:1 or 3:1 reflux:take-off ratio. The distillation was carried out until minimal material remained in the bottom of the still, with essentially all product distilled overhead. Periodically during the distillation, the overheads were collected, with 13 overall collections or cuts being taken in this experiment. For more detail about the size of each cut, and the quantification of the compounds in the overhead cuts by GC, see Table 3.

TABLE 3
Recovery of linear isomer as a function of cut combinations

| | | | | | Estimated | Estimated | Estimated | |
|---|---|---|---|---|---|---|---|---|
| | | g- | | wt. % | wt % | wt % | wt % | Recovery |
| Recovery | Physical | moles | grams | Linear | Branched | MMP | Double TFP | Linear |
| product | grams | Linear | Linear | (%) | (%) | (%) | (%) | (%) |
| Maximum Theoretical | — | 123.52 | 26707 | — | — | — | — | 100.0 |
| Recovery (100%) | | | | | | | | |
| Combine Cuts 5-12 | 27278 | 117.07 | 25312 | 92.8 | 6.8 | 0.2 | 0.0% | 94.6 |
| Combine Cuts 6-12 | 25477 | 111.69 | 24194 | 95.0 | 4.8 | 0.0 | 0.0% | 90.6 |
| Combine Cuts 7-12 | 23599 | 104.93 | 22687 | 96.1 | 3.6 | 0.0 | 0.0% | 64.9 |
| Combine Cuts 5-13 | 28330 | 121.70 | 26315 | 92.9 | 6.5 | 0.2 | 0.1% | 98.6 |
| Combine Cuts 6-13 | 26529 | 116.53 | 25196 | 95.0 | 4.6 | 0.0 | 0.1% | 94.3 |
| Combine Cuts 7-13 | 24851 | 109.56 | 23690 | 95.1 | 3.4 | 0.0 | 0.1% | 3.7 |
| Combine Cuts 8-13 | 20489 | 92.26 | 19948 | 97.4 | 2.1 | 0.0 | 0.1% | 74.7 |
| Combine Cuts 9-13 | 16045 | 73.27 | 15642 | 98.7 | 0.7 | 0.0 | 0.2% | 59.3 |
| Yield or linear product | — | 0.05 | 10 | . | — | . | — | 0.0 |
| lost to bottoms layer | | | | | | | | |

MMP = methylmercaptopropionate
TFP = trifluoropropene

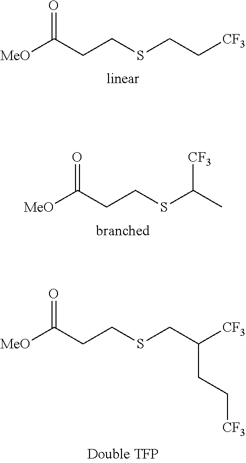 [SMILES: COC(=O)CCSCCC(F)(F)F ; COC(=O)CCSC(C)C(F)(F)F ; COC(=O)CCSCC(CCC(F)(F)F)C(F)(F)F]

### Example 4

A combination of branched isomer product overheads cuts were combined (21% branched, 76% linear isomer, 354 g) and charged to the 2 L still equipped with a 1″ diameter, 24 tray vacuum jacketed Oldershaw column equipped with a condenser and a tic-toc valve flow splitter, as shown in FIG. 4. The distillation was completed at 6-7 mmHg vacuum. Approximately 80 grams of material was distilled overhead in 3 cuts between 81.2° C. and 84.4° C. for the overheads temperature and 120.2° C. to 120.5° C. for the bottoms temperature. Upon removal of these overheads cuts, the bottom was analyzed by GC and found to have been enriched in linear isomer up to 93.4%, with only 5.3% branched remaining in the bottoms. The recovery of the linear isomer in the bottoms was 94.9% of the initial amount charged.

What is claimed is:

1. A process for preparing a compound of the formula II 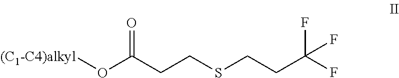
comprising contacting a compound of the formula I 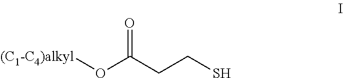
with 3,3,3-trifluoropropene in the presence of a compound of formula III 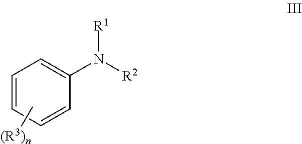
wherein R¹ and R² are each independently H or C₁-C₆ alkyl, wherein each hydrogen in C₁-C₆ alkyl is independently optionally substituted with Cl, Br, I, C₁-C₆ alkyl, or hydroxy; each R³ is independently C₁-C₆ alkyl, —NR⁴R⁵ or —NR⁴C(O)R⁵, wherein each R⁴ and R⁵ is independently C₁-C₆ alkyl, and n is an integer from 0 to 3; and a peroxide reagent of the formula IV or formula V 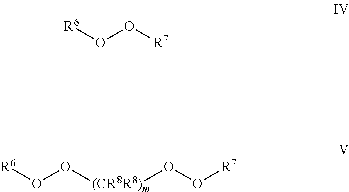
wherein R⁶ and R⁷ are each independently C(O)R⁹, C(R⁹)₃ or H, wherein each R⁸ and R⁹ is independently selected from H, C₁-C₆ alkyl, and C₆-C₁₀ aryl, provided that one of R⁶ or R⁷ is not H, and m is an integer from 2 to 4 to provide a compound of the formula II.

2. The process of claim 1, wherein the compound of formula III is selected from the group consisting of

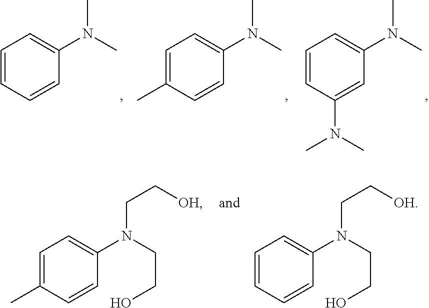

3. The process of claim 1, wherein the peroxide reagent is of the formula 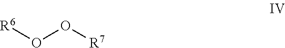
wherein R⁶ and R⁷ are each independently C(O)R⁹, C(R⁹)₃ or H, wherein each R⁹ is independently selected from H, C₁-C₆ alkyl, and C₆-C₁₀ aryl, and provided that one of R⁶ or R⁷ is not H.

4. The process of claim 3, wherein the peroxide reagent is selected from the group consisting of

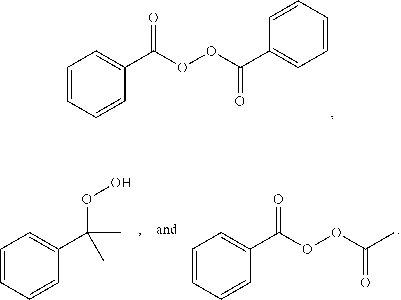 [SMILES: O=C(OOC(=O)c1ccccc1)c1ccccc1 ; CC(C)(OO)c1ccccc1 ; CC(=O)OOC(=O)c1ccccc1]

5. The process of claim 4 wherein the peroxide reagent is

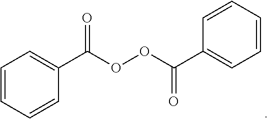 [SMILES: O=C(OOC(=O)c1ccccc1)c1ccccc1]

6. The process of claim 1, wherein the peroxide reagent is of the formula 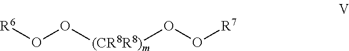
wherein R⁶ and R⁷ are each independently C(O)R⁹, C(R⁹)₃ or H, wherein each R⁸ and R⁹ is independently selected from H, C₁-C₆ alkyl, and C₆-C₁₀ aryl, provided that one of R⁶ or R⁷ is not H, and m is an integer from 2 to 4.

7. The process of claim 6, wherein the peroxide reagent is

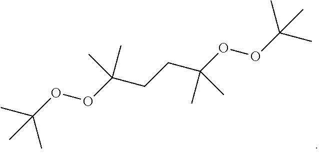 [SMILES: CC(C)(C)OOC(C)(C)CCC(C)(C)OOC(C)(C)C]

8. The process of claim 1, wherein the contacting is carried out in the presence of a solvent.

9. The process of claim 8, wherein the solvent is acetonitrile (CH₃CN), dioxane, N,N-dimethylformamide (DMF), dichloromethane (DCM), ethylacetate (EtOAc) N-methyl-2-pyrrolidone (NMP), tetrahydrofuran (THF), toluene or a mixture thereof.

10. The process of claim 1, wherein the process is carried out as a semi-batch process.

11. The process of claim 10, wherein the semi-batch process is carried out in a reaction vessel charged with a compound of formula III.

12. The process of claim 11, wherein the semi-batch process comprises a first feedstream comprising the peroxide reagent according to formula IV or formula V that is added to the reaction vessel over a period of time.

13. The process of claim 12, wherein the semi-batch process further comprises a second feedstream comprising 3,3,3-trifluoropropene that is added to the reaction vessel over a period of time.

14. The process of claim 13, wherein the compound of formula I is included in the first feedstream.

15. The process of claim 13, wherein the compound of formula I is included in a third feedstream.

16. The process of claim 13, wherein the first feedstream is added over a period of about 15 minutes to about 180 minutes and the second feedstream is added over a period of about 15 minutes to about 180 minutes.

17. The process of claim 13, wherein the compound of formula I is included in the reaction vessel prior to the introduction of either the first feedstream or the second feedstream.

18. The process of claim 11, wherein the reaction vessel is cooled to about 2° C. to about 20° C.

19. The process of claim 1, wherein the process is carried out as a continuous reactor process comprising a first feedstream comprising the compound according to formula III, a second feedstream comprising the peroxide reagent according to formula IV or V, a third feedstream comprising 3,3,3-trifluoropropene, or a combination thereof.

20. The process of claim 1, wherein the compound of formula II is purified by a multistage fractional distillation.
